(19) United States
(12) Patent Application Publication
(10) Pub. No.: US 2002/0065488 A1
(43) Pub. Date: May 30, 2002

(54) Needlepoint-covering member, method of assembling an injection needle with a needlepoint-covering member, and injection syringe with a needlepoint-covering member

(76) Inventors: Hitoshi Suzuki, Yokohama-shi (JP); Yousuke Mito, Yokohama-shi (JP)

Correspondence Address: KUBOVCIK & KUBOVCIK, SUITE 710, 900 17TH STREET NW, WASHINGTON, DC, 20006

(21) Appl. No.: 09/978,169

(22) Filed: Oct. 17, 2001

(30) Foreign Application Priority Data
Oct. 20, 2000 (JP) 2000-320429
Jan. 26, 2001 (JP) 2001-017721

(51) Int. Cl. A61M 5/32

(52) U.S. Cl. 604/192

(57) ABSTRACT

The present invention provides a novel needlepoint-covering member in which the flow of medical procedure is not disturbed before used as an injection syringe, the tip of the injection needle is suitably protected after use of the injection needle, and the protecting member cannot be removed easily even when external forces are exerted. A plate member whose end is fixed to the needle hub side and which can be bent to less than half of the axial length in the extended state in order to maintain the state before covering, and a covering portion for covering the needlepoint when the bent plate member is extended are provided, and the plate member has a length to allow the covering portion to reach the position of the needlepoint when extended. The plate member is widthwise concaved toward the needle for resisting external forces that tend to remove the covering portion that covers the needlepoint from the needlepoint.

### BACKGROUND OF THE INVENTION

1. Field of the Invention

The present invention relates to a needlepoint-covering member for covering a needlepoint of a used injection needle, and an injection syringe unit with the needlepoint-covering member. The present invention also relates to a technical creation that also serves as a catheter safeguard assembly that can be attached with a catheter outside the needle.

2. Description of the Related Art

A used injection needle has a hazard of transmitting AIDS, hepatitis, etc. In order to avoid the hazard, various injection syringes having a protector for preventing the tip of the used injection needle from accidental contact are provided.

The injection syringes are now classified for description.

The first type is the one in which the whole needle is stored in a protective tube provided around an outer periphery of an outer tube of the injection syringe by pushing the protective tube toward the needlepoint after use.

Many of the protective tubes of this type are heavy to slide, and thus a medical worker has to use one hand for fixing the outer tube and another hand for pushing the protective tube. At the same time, however, in the process of injection, it is necessary to press the needled portion after the needle is withdrawn in many cases. Therefore, the procedure to protect the needle that uses both hands tends to be left over, which leaves the hazard of accident.

As one of techniques to reduce the hazard, there is a type having a protector connected to a needle hub via a hinge or the like. The injection syringes of this type are now further classified by mechanism.

Examples of the injection syringe provided with a mechanism for moving the protector from the position before use to the position after use include the syringe provided with a coil spring mechanism as disclosed in JP-A-03-139363 and JP-U-08-86744, and the syringe provided with a leaf spring mechanism as disclosed in JP-A-08-112348 (see FIGS. 34 to 36).

Examples of the injection syringe provided with a mechanism for moving the protector from the position before use to the appropriate position after use include the syringe provided with a string long enough to reach the appropriate position disclosed in JP-A-10-272182 and JP-A-03-139363, and the syringe employing a link mechanism disclosed in JP-A-07-250898 and JP-A-08-206204.

Examples of the injection syringe provided with a mechanism for preventing a reversal movement of the protector after use to avoid the exposure of the needlepoint include the syringes disclosed in JP-A-03-504205, JP-A-07-250898 (see FIG. 35), and JP-A-08-206204 (see FIG. 36).

There are some other types of injection syringes that employ easier construction than the syringes described above, such as the syringe employing a tube disclosed in JP-A-08-107932 and the syringe employing a flexible cap in which the portion corresponding to the needlepoint is of non-breakable structure disclosed in JP-U-64-28652.

As seen from the description of the related art, it is understood that the functions to ensure the state that does not disturb the flow of medical procedure before it is used as an injection needle, to move the protector to the adequate position after use of the injection needle, and to prevent the protector from being removed even when external forces are exerted after the needlepoint is covered by the protector are required.

### SUMMARY OF THE INVENTION

It is an object of the present invention to provide a novel needlepoint-covering member that does not disturb the medical procedure before it is used as an injection needle, protects the tip of the injection needle adequately after the injection needle is used, and prevents the protecting member from being detached easily even when external forces are exerted.

It is also an object of the present invention to provide a method of assembling the injection needle with a needlepoint-covering member.

It is also an object of the present invention not only to provide an injection needle with a novel needlepoint-covering member that does not disturb the medical procedure before it is used as an injection needle, protects the tip of the injection needle adequately after the injection needle is used, and prevents the protecting member from being detached easily even when external forces are exerted, but also to provide an injection syringe with a novel needlepoint-covering member that does not disturb the medical procedure before it is used as an injection needle, protects the tip of the injection needle adequately after the injection needle is used, and prevents the protecting member from being detached easily even when external forces are exerted.

In order to solve the problems described above, this application provides the invention as described below.

The present invention provides a needlepoint-covering member for covering the needlepoint (1a) of the injection needle (20) comprising a needle (1) having a sharp needlepoint (1a) and a needle hub (2) provided at the end of the needle (1) far from the needlepoint (1a) for being joined with the cylinder, comprising: a plate member (4) whose end is fixed to the needle hub (2) side and which can be bent to less than half of the axial length in the extended state in order to maintain the state before covering; and a covering portion (e.g. a sleeve (6)) for covering the needlepoint (1a) when the bent plate member (4) is extended; wherein the plate member (4) has a length to allow the covering portion (6) to reach the position of the needlepoint (1a) when extended, and is widthwise concaved toward the needle (1) for resisting external forces that tend to remove the covering portion (6) that covers the needlepoint (1a) from the needlepoint (1a).

The term “sharp needlepoint (1a)” here means the portion formed by cutting the tip of the tubular needle (1) diagonally with respect to the axial direction for being stuck to a patient. Hereinafter, the needlepoint (1a) side means the side of the patient and the proximal side means the side closer to the medical worker, or the far side from the needlepoint (1a) side.

The term “needle hub (2)” is not limited to the needle hub (2) of the disposable injection needle, and it may be an internal needle hub (2) in the catheter assembly, an outer tube of the injection syringe, or even the root portion of a winged needle.

The term “plate member (4)” may be a metal leaf spring, a thin resin plate, non-woven fabric, laminate paper, etc.

The expression that an end of the plate member (4) is “fixed to the needle hub (2) side” includes the case in which the end is fixed to the needle hub (2) and the case in which the end is fixed to the syringe to which the needle hub (2) is fixed.

The expression that the plate member (4) can be “bent” means to be continuously deformable between a state in which no external force is exerted on the plate member (4) and a “bent state” in which the end of the plate member (4) on the needlepoint (1a) side stays away from the needlepoint (1a). It is not necessary to provide the portion that can be bent all over the plate member (4), and the portion must simply be provided at a part of the plate member (4). This also applies to other aspects of the present invention.

In the “bent state”, the needlepoint (1a) is exposed, which is a state suitable for being stuck to the patient.

The term “less than half of the axial length in the extended state” regarding the plate member (4) is applied within the range of the portion of the plate member (4) that is practically deformable. For example, in the case where the portion of the plate member (4) behind the sleeve (6) cannot be deformed as in the case of the needlepoint-covering member shown in FIG. 7, it means that the axial length of the plate member (4) projecting from the sleeve (6) toward the needlepoint (1a) side is less than half of that in the extended state. The axial length of the plate member (4) when being bent means the length in the direction of the axis of the needle (1) as shown by “L” in FIG. 8. These are also applied to other aspects of the invention.

According to the present invention, before use of the injection needle (20), the plate member (4) having a axial length of the needle (1) less than half of that in the extended state is in the bent state, and the needlepoint (1a) is exposed and is ready for use as an injection needle (20).

After usage of the injection needle (20), the plate member (4) in the bent state is extended. Then, the covering portion (6) reaches the position of the needlepoint (1a).

After the needlepoint (1a) is covered by the covering portion (6), the plate member (4) being widthwise concaved toward the needle (1) resists external forces that tend to remove the covering portion (6) from the needlepoint (1a) (external forces that tend to move the covering portion (6) toward the needle hub (2) side), and thus the needlepoint (1a) is maintained in the state of being covered by the covering portion (6). Therefore, it contributes to protect the person who operates the injection syringe (20) from the needlepoint (1a).

The present invention may further restrict the needlepoint-covering member.

According to the present invention, the plate member (4) is preferably provided with a wrap-around fixing portion (4e) for embracing the circumference of the needle (1) and fixing the needle (1) in the vicinity of the center in the axial direction of the plate member (4). The wrap-around fixing portion (4e) is a needlepoint-covering member formed in such a manner that, when it embraces and fixes the circumference of the needle (1), the curvature radius of the plate member (4) that is widthwise concaved toward the needle (1) is decreased.

Regarding the plate member (4), the term “widthwise concaved toward the needle (1)” includes the case where it is curved in a U-shape in cross section perpendicularly with respect to the longitudinal direction of the plate member (4), and the case where it is formed in a V-shaped, or an angular C-shaped convex.

Therefore, in this invention, after the needlepoint (1a) is covered by the covering portion (6), the wrap-around fixing portion (4e) can enclose and fix the circumference of the needle (1) in the vicinity of the axial center of the needle (1). Then, the wrap-around fixing portion (4e) reduces the curvature radius of the curve of the plate member (4) that is widthwise concaved toward the needle (1) and thus geometrical moment of inertia resisting against the external force that tends to remove the covering portion (6) from the needlepoint (1a) increases, so that the needlepoint (1a) is remained in the state of being covered by the covering portion (6). In addition, since the needle (1) and the plate member (4) are unified by the wrap-around fixing portion (4e), the feeling of security for the needle (1) and the needlepoint (1a) after use increases as well.

Since the curvature radius of the plate member (4) is not reduced yet at the moment before wrap-around fixing the circumference of the needle (1) by the wrap-around fixing portion (4e), it does not hinder the covering portion (6) from covering the needlepoint (1a).

The present invention may further restrict the needlepoint-covering member.

According to the present invention, preferably, the covering portion (6) comprises an insertion hole (6a) through which the needle (1) and the plate member (4) are inserted, and a engaging portion (6c) for engaging with an engaged portion (4b) on the plate member (4) in a state in which the covering portion (6) reaches the needlepoint (1a); wherein the plate member (4) comprises the engaged portion (4b) to be engaged with the engaging portion (6c).

Therefore, according to the present invention, the needle (1) and the plate member (4) is inserted through the insertion hole (6a) and the engaging portion (6c) engages the engaged portion (4b) of the plate member (4) in a state in which the covering portion (6) reaches the needlepoint (1a). Accordingly, even when external forces are exerted, the covering portion (6) does not come apart from the needle (1) and thus the needlepoint (1a) resists being exposed unless the engagement is released.

The invention according to the present application provides a needlepoint-covering member for covering the needlepoint (1a) of the injection needle (20) comprising a needle (1) having a sharp needlepoint (1a) and a needle hub (2) provided at the end of the needle (1) far from the needlepoint (1a) for being joined with the cylinder, comprising: a plate member (4) whose end is fixed to the needle hub (2) side and which can be bent to less than half of the axial length in the extended state in order to maintain the state before covering; and a covering portion (e.g. a sleeve 6) for covering the needlepoint (1a) when the bent plate member (4) is extended; wherein the covering portion (6) has a tubular shape for allowing the needle (1) to pass through when the plate member (4) is bent; the plate member (4) has a length to allow the covering portion (6) to reach the position of the needlepoint (1a) when extended; and the direction in which the plate member (4) is bent by external forces that tend to remove the covering portion (6) from the needlepoint (1a) is convex toward the needle (1).

In the present invention, since the needle (1) passes through the tubular covering portion (6), and the plate member (4) is in a bent state that its length in the direction of the axis of the needle (1) is less than half of that in the extended state before use of the injection needle (20), the needlepoint (1a) is exposed and is ready for use as an injection needle (20).

After usage of the injection needle (20), the plate member (4) in the bent state is extended. Then the covering portion (6) reaches the position of the needlepoint (1a).

After the needlepoint (1a) is covered by the covering portion (6), when external forces that tend to remove the covering portion (6) from the needlepoint (1a) so increases that it bends the extended plate member (4), the bending direction control portion forces the plate member (4) to be bent convexly toward the needle (1). Then the plate member (4) abuts against the needle (1) where it cannot be bent anymore, and thus the needlepoint (1a) is maintained in the state of being covered by the covering portion (6).

In the present invention, preferably, the plate member (4) is a needlepoint-covering member wherein the bending direction control portion is formed of a plate member constructed so as to be bent widthwise convexly toward the needle (1).

According to the needlepoint-covering member of the present invention, preferably, geometrical moment of inertia of at least one of both ends of the plate member (4) in the extended state is determined to the value lower than that of other portions.

As specific means to reduce the “geometrical moment of inertia”, for example, a narrow portion (4f) having a narrower width may be formed on the plate member (4). Since the plate member (4) that is curved widthwise along its whole length is employed, even when the curvature radius is increased in a particular part, the geometrical moment of inertia at the part maybe reduced. The geometrical moment of inertia can be reduced by reducing the thickness of the plate member (4) as well.

According to the theoretical expression of the threshold value of buckling, the buckling weight is inversely proportional to the square value of the length of the member, and is proportional to the geometrical moment of inertia. Therefore, reduction of the geometrical moment of inertia at both ends of the plate member (4) contributes to reduce the possibility of bending at other portions having larger geometrical moment of inertia.

Therefore, according to the present invention, since geometrical moment of inertia of at least one of both ends of the plate member (4) in the extended state is reduced, the portion tends to bend convexly toward the needle (1). When the portion bends, the curvature radius of the curve of the plate member (4) being curved convexly in the direction of the width with respect to the needle (1) decreases, but the widthwise curvature of other portions is maintained. Simultaneously, the plate member (4) abuts against the needle (1), and thus it cannot be bent anymore. Therefore, the needlepoint (1a) is maintained in the state of being covered by the covering portion (6).

Preferably, the needlepoint-covering member according to the present invention comprises a needlepoint hinge (10a) for rotatably connecting the covering portion (6) and the plate member (4) on the needlepoint (1a) side of the plate member (4).

The expression “rotatably connecting the covering portion (6) and the plate member (4) on the needlepoint (1a) side of the plate member (4)” regarding the “needlepoint hinge (10a)” means that the needle hinge (10a) is rotatably disposed on the side far from the needle (1) with respect to the extension of the plate member (4) as shown in FIGS. 23(b) and (c).

Therefore, when external forces are exerted to the plate member (4) in the extended state in the direction of generation of bending, the existence of the needlepoint hinge (10a) produces torque in the direction toward the needle (1) on the plate member (4), so that the plate member (4) is bent and deformed convexly toward the needle (1). Then, the bent plate member (4) is brought into contact with the needle (1) and thus it cannot be bent any more, and the needlepoint (1a) is maintained in the state being covered by the covering portion (6).

According to the present invention, preferably, the needlepoint-covering member comprises a proximal hinge (10b) for rotatably connecting the needle hub (2) and the plate member (4) on the side far from the needle (1) with respect to the plate member (4).

The expression “rotatably connecting the needle hub (2) and the plate member (4) on the side far from the needle (1) with respect to the plate member (4)” means that the proximal hinge (10b) is rotatably disposed on the side far from the needle (1) with respect to the extension of the plate member (4), as shown in FIGS. 23(b) and (c).

Therefore, according to the invention, when an external force in the direction of generation of bending is exerted on the plate member (4) in the extended state, existence of the proximal hinge (10b) produces torque in the direction toward the needle (1) on the plate member (4), so that the plate member (4) is bent and deformed convexly toward the needle (1). Then, the bent plate member (4) is brought into contact with the needle (1) and thus it cannot be bent any more, and the needlepoint (1a) is maintained in the state being covered by the covering portion (6).

According to present invention, preferably, the needlepoint-covering member comprises bending-position-maintaining members (8c, 8d) for maintaining the plate member (4) in the bent state before covering the needlepoint (1a) by the covering portion (6), and a trigger-function portion for releasing a load from the state of being maintained in the bent state.

The “bending-position-maintaining member” includes a pair of engaging members, a member for restraining the plate member (4) in the bent state, etc. A pair of engaging members includes not only a limitation of “a proximal hook (8d) and needlepoint hook (8c)”, but also a proximal hook formed integrally with the plate member (4) or the needle hub (2), or a needlepoint hook formed integrally with a member constructing the plate member (4) or the covering portion (6).

The “trigger-function portion” means a portion for achieving the function of releasing the bent state maintained by the bending-position-maintaining member. It may be provided as an independent portion, or as a portion performing the trigger function by using a part of the plate member (4) in the bent state.

In the invention, the needlepoint (1a) is not covered by the covering portion (6) before using the needle (1). In this state, the bending-position-maintaining members (8c, 8d) maintain the plate member (4) in the bent state. After the needle (1) is used, or when the needlepoint (1a) is required to be covered, the trigger-function portion is actuated to release the state maintained by the bending-position-maintaining members (8c, 8d) into the unloaded state. Then, the plate member (4) is extended and the covering portion (6) covers the needlepoint (1a). Therefore, the operation after use of the needle (1) is easily made.

According to the present invention, preferably, the plate member (4) comprises a proximal hinge (10b) for fixing the end on the needle hub (2) side of the plate member (4) so as to be capable of rotating with respect to the axis of the needle (1), an upwardly opened hook-shaped proximal hook (8d) fixed on the plate member (4) at the position closer to the needle hub (2), and a downwardly opened hook-shaped needlepoint hook (8c) fixed on the plate member (4) at the position closer to the needlepoint (1a); wherein the plate member (4) is maintained in a bent state by engaging the proximal hook (8d) and the needlepoint hook (8c); and the engaged state between the proximal hook (8d) and the needlepoint hook (8c) may be released by changing the angle of the proximal hinge (10b).

According to the present invention, the plate member (4) is fixed at its end portion on the needle hub (2) side by the proximal hinge (10b) so as to be capable of rotating with respect to the axis of the needle (1). Therefore, even when the plate member (4) is in the bent state or in the extended state, the end portion of the plate member (4) is fixed to the needle hub (2) side.

In the bent state, the plate member (4) is maintained in the bent state by the upwardly opened hook-shaped proximal hook (8d) and the downwardly opened hook-shaped needlepoint hook (8c) engaged with each other.

In order to extend the plate member (4) in the bent state after use of the needle (1), the angle of the proximal hinge (10b) is changed by moving the plate member (4). Then the engagement between the proximal hook (8d) and the needlepoint hook (8c) is released and the plate member (4) extends.

In the needlepoint-covering member according to the present invention, preferably, the plate member (4) comprises a needlepoint hinge (10a) for fixing the end portion on the needlepoint (1a) side of the plate member (4) so as to be capable of rotating with respect to the axis of the needle (1).

In this invention, since the plate member (4) is fixed with the needlepoint hinge (10a) and is capable of rotating the end on the needlepoint (1a) side with respect to the axis of the needle (1), transition from the bent state to the extended state can be performed smoothly.

According to the present invention, preferably, the needlepoint-covering member comprises a cap (11) for covering at least the needlepoint (1a) and the needle (1) with the plate member (4) maintained in the bent state.

In this invention, since the cap (11) covers at least the needlepoint (1a) and the needle (1) with the plate member (4) maintained in the bent state, the sanitary conditions of the needlepoint (1a) and the needle (1) before use may be maintained.

The present invention also relates to a method of assembling an injection needle with a needlepoint-covering member comprising a fixing step for fixing a needle (1) and a needle hub (2) for manufacturing a hub with a needle, an incorporating step for centering and incorporating the needlepoint-covering member into a cap (11) for covering the needle (1), and a capping step for incorporating the hub with a needle into the cap (11) that is provided with the needlepoint-covering member incorporated in the incorporating step.

The present invention also relates to an injection syringe with a needlepoint-covering member comprising a hub with a needle in which a needle (1) and a needle hub (2) are fixed with each other, and an injection syringe provided with the hub with a needle fixed at its tip.

As is described thus far, the present invention provides a novel needlepoint-covering member in which the flow of medical procedure is not disturbed before use as an injection needle, in which the tip of the injection needle is suitably protected after usage of the injection needle, and in which the protecting member cannot be removed easily even when external forces are exerted.

The present invention further provides a method of assembling an injection needle with a needlepoint-covering member.

The present invention further provides a novel injection needle with a needlepoint-covering member in which the flow of medical procedure is not disturbed before use as a injection needle, in which the tip of the injection needle is suitably protected after usage of the injection needle, and in which the protecting member cannot be removed easily even when external forces are exerted.

Though the invention or the portion disclosed in JP-A-03-139363 and JP-U-06-86744 comprise a coil spring mechanism, and those disclosed in JP-A-08-107932 or JP-U-64-28652 comprise a tube or a cap with a notch, they are not provided with a plate member as in the present invention.

Though the inventions disclosed in JP-A-10-272182 and JP-A-03-139363 comprises a string as long as it reaches the adequate position for moving the portion or the member for protecting the needlepoint to the adequate position or for fixing them at that position, and the invention disclosed in JP-A-07-250898 and JP-A-08-206204 employ a link mechanism, the present invention differs from these inventions in that the plate member having an adequate length is employed.

Though the invention disclosed in JP-A-03-504205, JP-A-07-250898 (see FIG. 35), and JP-A-08-206204 (see FIG. 36) comprise a mechanism for preventing the reversal movement of the protector after use so that the needlepoint is not exposed, the present invention differs from these inventions in that a construction in which the plate member after protecting the needlepoint resists bending is employed.

Though the invention disclosed in JP-A-08-112348 (see FIG. 34) comprises a leaf spring mechanism as in the present invention, the present invention differs from the invention in that the construction for making the plate member resist bending is achieved by adoption of the cross sectional configuration of the plate member or by utilizing the needle for making the plate member resist bending against external forces.

### BRIEF DESCRIPTION OF THE DRAWINGS

FIG. 1 is a perspective view showing a state in use in the first embodiment of the present invention.

FIG. 2 is a cross sectional view of a plate member at a site where the plate member is bent.

FIG. 3 is a cross sectional view of the plate member after use at a site where the plate member is bent in use.

FIG. 4 is an assembled perspective view showing a state after use.

FIG. 5 is a perspective view showing a state in use in the second embodiment of the present invention.

FIG. 6 is an assembled perspective view showing a state after use.

FIG. 7 is an assembled perspective view showing a state in use in the other embodiment.

FIG. 8 is a perspective view showing a state in use in the third embodiment of the present invention.

FIG. 9 is an assembled perspective view showing a state after use.

FIG. 10 contains cross sectional views comparing a bent state of a plate member .

FIG. 11 is a perspective view showing a state in use in the other embodiment.

FIG. 12 is a perspective view showing a state in use in the fourth embodiment.

FIG. 13 is a perspective view showing a state in use in the fifth embodiment.

FIG. 14 is a perspective view showing a state in use in the sixth embodiment of the present invention.

FIG. 15 contains cross sectional views showing a state in which the frictional engagement is released immediately after use.

FIG. 16 is a perspective view showing a state after use.

FIG. 17 is a perspective view showing a state in which a cap is mounted.

FIG. 18 is a perspective view showing a bending mechanism before assembly.

FIG. 19 is a perspective view of a conventional injection needle and an outer tube of an injection cylinder.

FIG. 20 contains side sectional views showing a variation of a triggering mechanism.

FIG. 21 contains side sectional views showing the other variation of a triggering mechanism.

FIG. 22 is a perspective view showing the seventh embodiment.

FIG. 23 contains cross sectional views showing the seventh embodiment.

FIG. 24 contains side views showing the seventh embodiment.

FIG. 25 is a perspective view showing a modification of the seventh embodiment.

FIG. 26 is aside view showing a modification of the seventh embodiment.

FIG. 27 contains perspective views showing a modification of the eighth embodiment.

FIG. 28 contains perspective views showing the eight embodiment.

FIG. 29 contains perspective views showing a modification of the eighth embodiment.

FIG. 30 contains perspective views showing the assembling procedure.

FIG. 31 contains perspective views showing a modification of the ninth embodiment.

FIG. 32 contains perspective views showing the ninth embodiment.

FIG. 33 contains perspective views showing a modification of the ninth embodiment.

FIG. 34 contains representative drawings in JP-A-08-112348 comprising a leaf spring mechanism.

FIG. 35 contains representative drawings in JP-A-07-250898 comprising a reversal movement preventing mechanism so as to prevent a needlepoint from being exposed.

FIG. 36 contains representative drawings in JP-A-08-206204 comprising a reversal movement preventing mechanism so as to prevent a needlepoint from being exposed.

### DESCRIPTION OF THE PREFERRED EMBODIMENTS

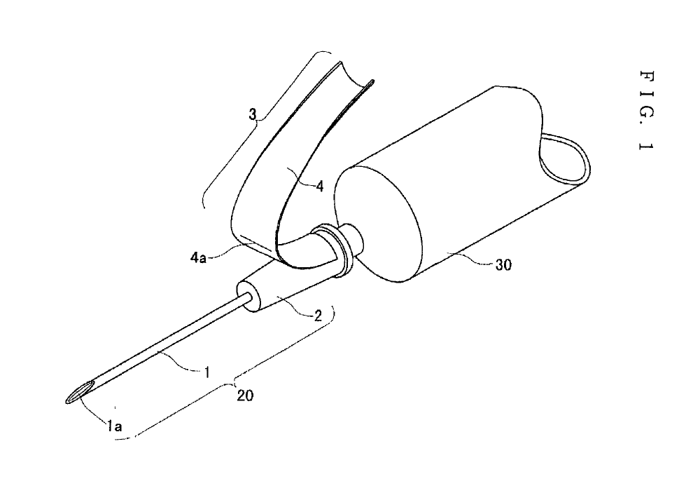

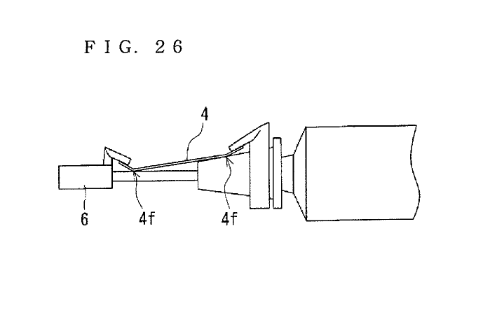

Referring now to the embodiments and drawings, the present invention will be described in detail. The drawings used here are FIGS. 1 to 33.

FIGS. 1 to 4 show the first embodiment of the present invention.

This embodiment is specifically suited for an injection needle (20) whose needle (1) is as short as 13 mm or 16 mm.

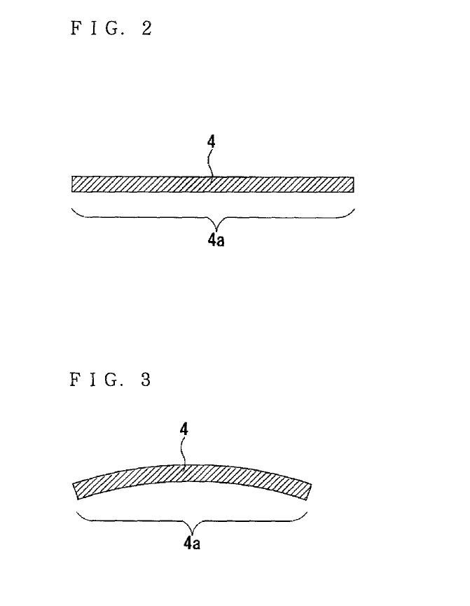

FIG. 1 shows an injection syringe immediately before being stuck through the patient's skin. The injection syringe comprises an injection needle (20) including a needle hub (2) fixed with the proximal end of a needle (1) having a sharp tip, and an outer tube (30) of the injection syringe connected to the injection needle (20). Though a plate member (4) made of resin and having a cross section corresponding to the curvature of the side surface of the needle hub (2) extends from the side surface, the plate member (4) is bent at the position closer to the needle hub (2) before sticking. Therefore, the needle (1) and the needlepoint (1a) are exposed, and thus the sticking procedure is not disturbed. By selecting adequate curvature, width and thickness for the bending mechanism (3), the cross section along the bent line (4a) exhibits a flat shape as shown in FIG. 2, whereby preventing formation of creases. In addition, corners of the plate member (4) are chamfered in order to protect the medical worker's finger against injury through handling.

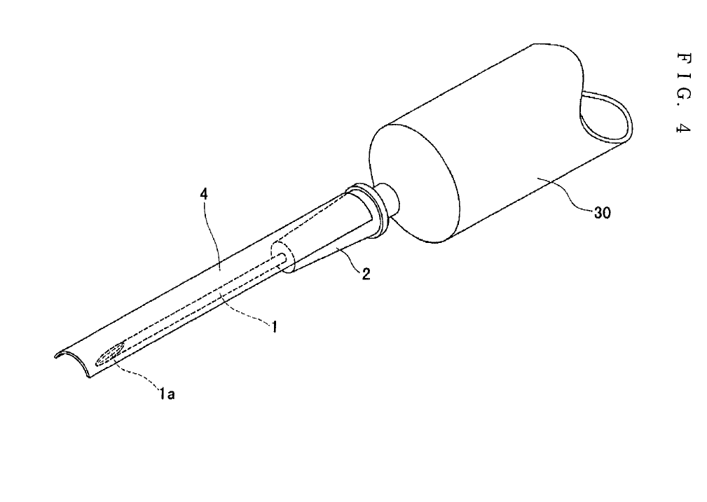

After use, the medical worker withdraws the injection needle (20) from the patient, and pushes the tip portion of the plate member (4) from the bent state to the extending direction to the extended state by a finger or the like. Then, the cross section of the plate member (4) at the bent position (4a), which has been rectangular, deforms into the curved cross section as shown in FIG. 3 by the influence of the curvature of the needle hub (2). This operation does not require too much force. As a consequent, the cross section of the plate member (4) is convex toward the needle (1) from the proximal side to the needlepoint (1a) as shown in FIG. 4, and the needle (1) and the needlepoint (1a) is stored in the internal space formed by this curvature. In addition, geometrical moment of inertia increases due to the curvature, thereby preventing formation of creases that may cause concentration of stresses. Therefore, the plate member (4) containing the used injection needle therein can hardly be deformed by external forces, and thus the used needle can be maintained in the state being stored.

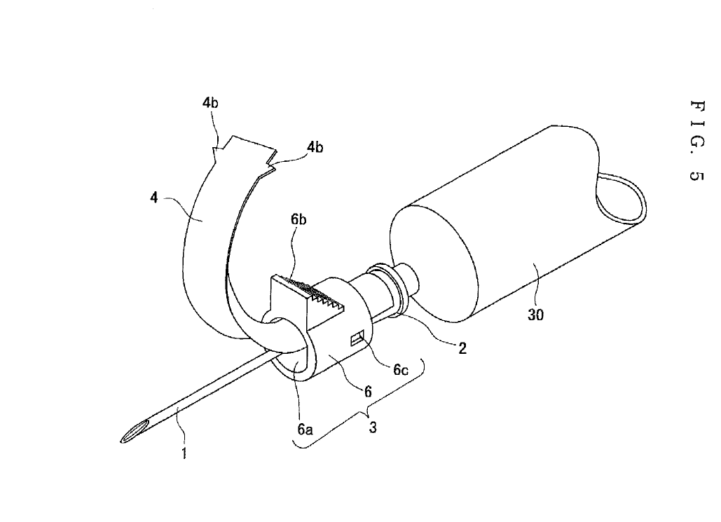

FIGS. 5 to 7 show the second embodiment of the present invention.

FIG. 5 is an injection syringe immediately before being stuck into the patient's skin. An end of the plate member (4) bent away from the needlepoint (1a) convexly toward the needlepoint (1a) to maintain the state before covering is fixed to the needle hub (2).

There is provided a tubular sleeve (6) for covering the needlepoint (1a) when the plate member (4) in the bent state is extended. The sleeve (6) comprises an insertion hole (6a) through which the needle (1) and the plate member (4) are inserted, an engaging portion (6c) for engaging with the plate member (4) in a state in which the sleeve (6) reaches the needlepoint (1a), and a knob (6b) to put the finger on when moving the sleeve (6). On the other hand, the plate member (4) is provided with an engaged portion (4b) projecting to be engaged with the engaging portion (6c).

The covering portion (6) is preferably provided with an inner wall surface that decreases the curvature radius of the plate member (4) that is widthwise concaving with respect to the needle (1).

Therefore, when the needlepoint (1a) is covered with the covering portion (6), the inner wall surface of the covering portion (6) decreases the curvature radius of the plate member (4) in the direction of the width. Therefore, it can hardly deformed by external forces, and thus the needlepoint (1a) is prevented from being easily exposed.

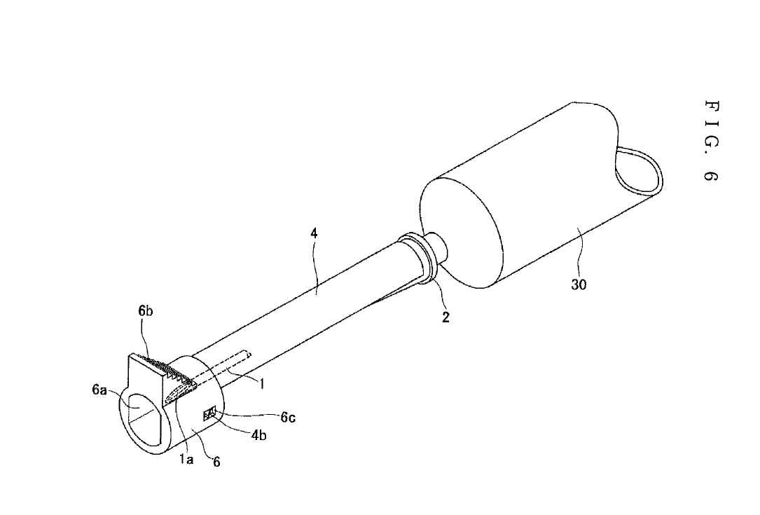

FIG. 6 shows the state after use. The medical worker withdraws the injection needle (20) from the patient, and pushes the knob (6b) on the sleeve (6) toward the needlepoint (1a) with his/her finger or the like. Then, the plate member (4) restricted widthwise by the sleeve (6) extends along with passage of the sleeve (6). Then, when the sleeve (6) reaches the position of storing the needlepoint (1a), the engaging portion (6c) and the engaged portion (4b) of the plate member (4) engage each other and the needlepoint (1a) is stored in the sleeve (6). Concurrently, the plate member (4) curved in widthwise cross section all over from the proximal side to the needlepoint side. As a consequent, since the plate member (4) containing the used injection needle therein can hardly be deformed by external forces and thus the needlepoint (1a) is maintained in the state being stored in the sleeve (6), the used needle is prevented from being exposed again.

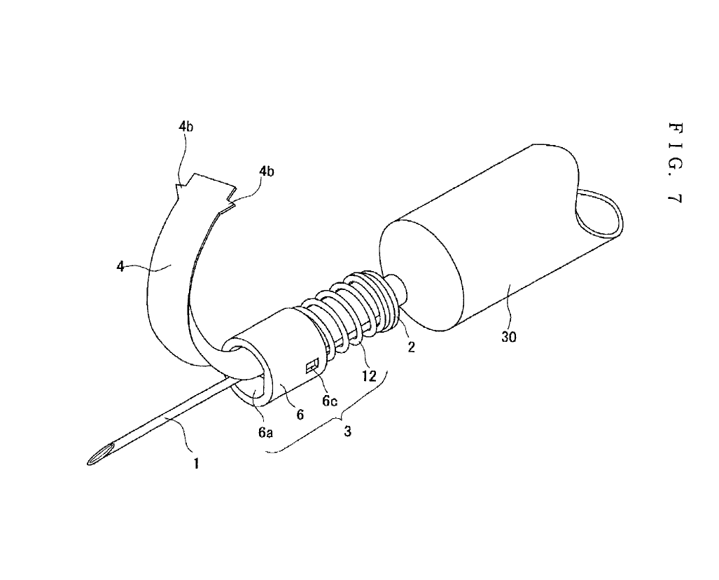

FIG. 7 is a modification of the embodiment shown in FIG. 5.

In this modification, the needlepoint-covering member is provided with urging means for urging the covering portion (6) toward the needlepoint (1a).

Since the urging means is provided, the covering portion (6) moves toward the needlepoint (1a) by bringing the urging means into action. Therefore, the operation of the needle (1) after use can be performed easily.

In this example, a spring (12) is disposed between the sleeve (6) and the needle hub (2), and a trigger (not shown) for releasing the spring (12) which is mounted in a compressed state is provided, so that the sleeve (6) is pushed toward the needlepoint side by the urging force of the spring (12). This modification ensures easier movement of the sleeve (6) in comparison with the case of providing the knob (6b) to move manually as shown in FIGS. 5 and 6.

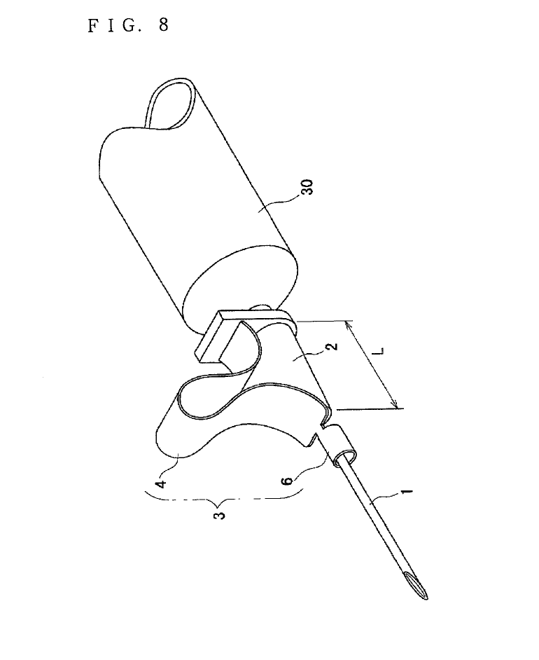

FIGS. 8 to 11 show the third embodiment.

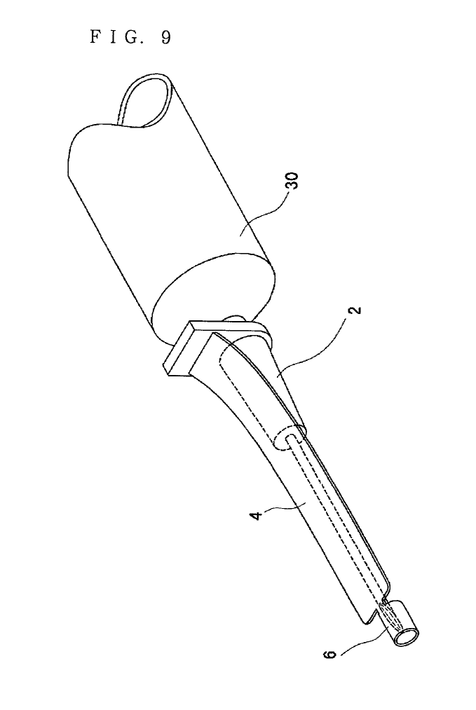

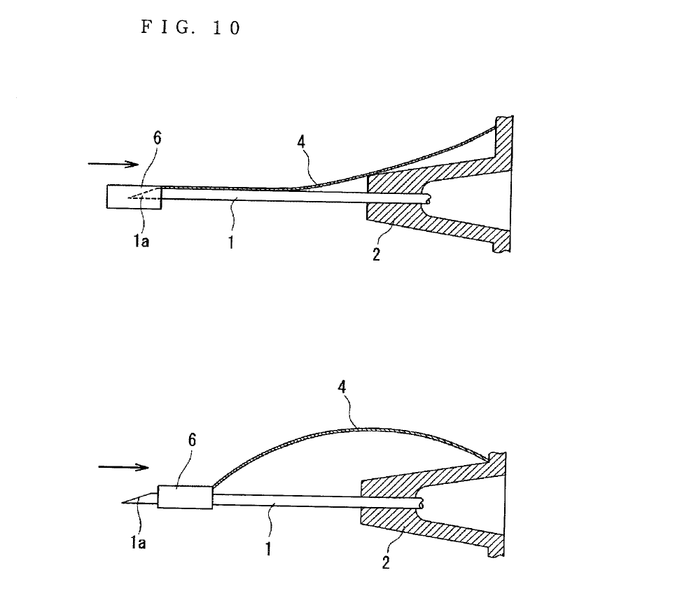

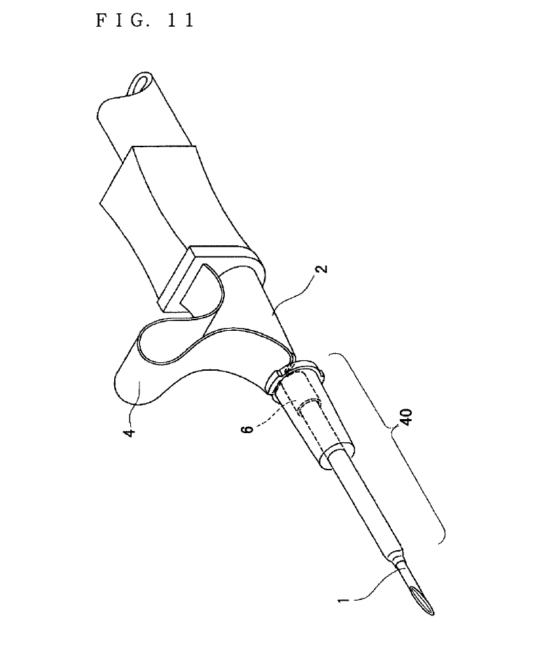

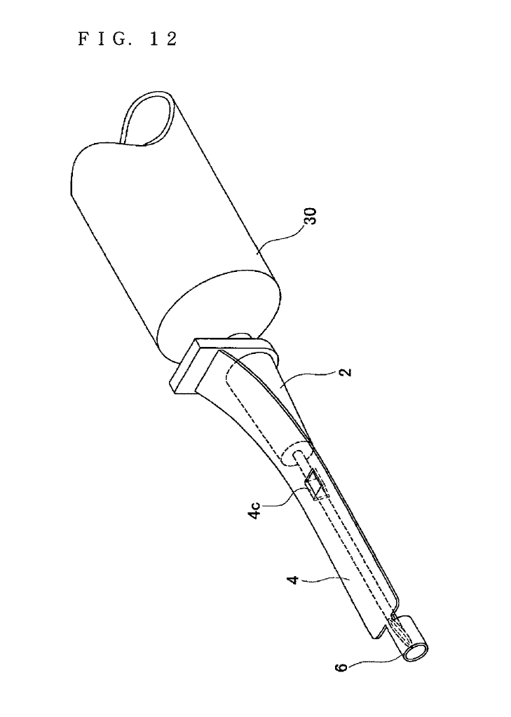

FIG. 8 shows the state immediately before being stuck into the patient's skin, and FIG. 9 shows a state in which the needlepoint (1a) is protected after being stuck. FIG. 10 is a drawing explaining a case in which external forces are exerted after the needlepoint (1a) is protected. FIG. 11 is a drawing showing a modification in which a catheter (40) is disposed outside the needle (1) to construct a catheter safeguard assembly.

The plate member (4) made of metal plate is fixed above the needle hub (2) in such a manner that the proximal side of the plate member (4) is oriented at an angle intersecting the needle (1). The plate member (4) is provided at its another end with a tubular sleeve (6) continuously from the plate member (4). The sleeve (6) is placed in the vicinity of the needle hub (2) with the needle (1) inserted.

After use, the medical worker withdraws the injection needle (20) from the patient, and pushes the bent portion of the plate member (4) with his/her finger from the bent state to the extending direction. Then, the plate member (4) guided by the sleeve (6) extends to the position where the sleeve (6) stores the needlepoint (1a). Since the proximal side of the plate member (4) is fixed at an angle of intersecting the needle (1), the plate member (4) is longitudinally deformed into convex toward the needle (1) as shown in FIG. 9.

As shown in FIG. 10, the plate member (4) deformed convexly toward the needle (1) as shown in FIG. 9 can hardly be deformed by external forces that tend to push the sleeve (6) toward the needle hub (2), and thus the needlepoint (1a) is maintained in the state being stored in the sleeve (6) and prevented from being exposed, in comparison with the case where it is concaved toward the needle (1).

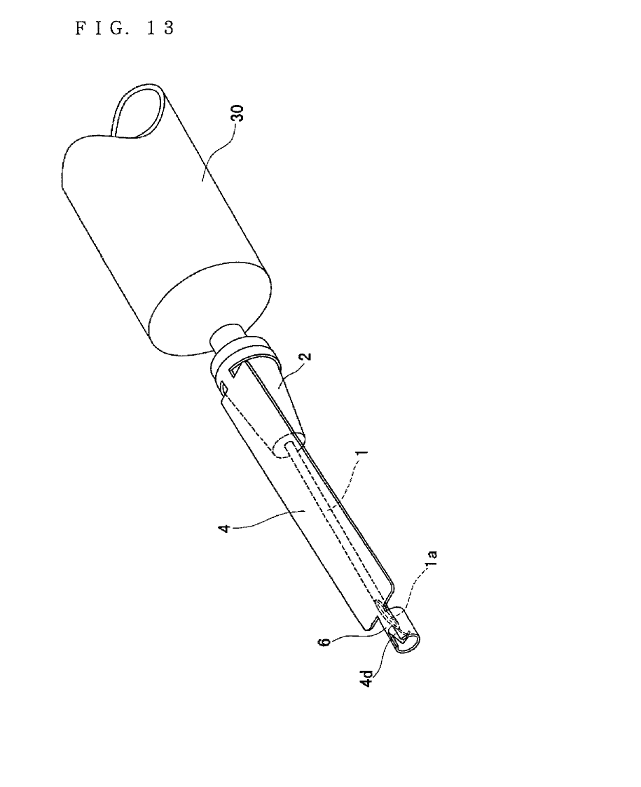

FIG. 12 shows the fourth embodiment.

In this embodiment, the plate member (4) is provided with a needle-holding portion (4c) to be engaged with the needle (1) after the needlepoint (1a) is covered by the sleeve (6) at the longitudinal midsection of the plate member (4). After the needlepoint (1a) is covered by the sleeve (6), the needle-holding portion (4c) provided at the longitudinal midsection of the plate member (4) engages with the needle (1). Therefore, even when external forces that tend to push the sleeve (6) toward the needle hub (2) are exerted, the sleeve (6) will never be drawn away from the needle (1) unless the engagement between them is released, and thus the needlepoint (1a) is prevented from being exposed easily.

FIG. 13 shows the fifth embodiment.

In this embodiment, the sleeve (6) is provided with a needlepoint-holding portion (4d) that comes close to the needlepoint (1a) when the needlepoint (1a) is covered by the sleeve (6) for preventing the needlepoint (1a) from being projected from the sleeve (6) after being covered. Though the needlepoint-holding portion (4d) that serves as a reversal-movement-preventing hook presses the side surface of the needle (1) when the plate member (4) is bent, the pressing force does not exert any influence on the sliding movement. With the needlepoint-holding portion (4d), the needlepoint (1a) cannot be exposed easily even with external forces that tend to push the sleeve (6) toward the needle hub (2). In addition, since the needlepoint-holding portion (4d) is formed by processing a part of the plate member (4), it can be supplied at low cost.

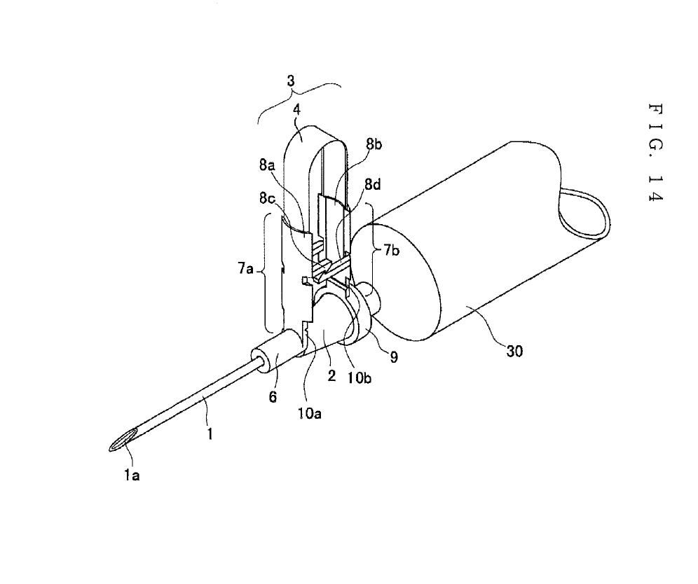

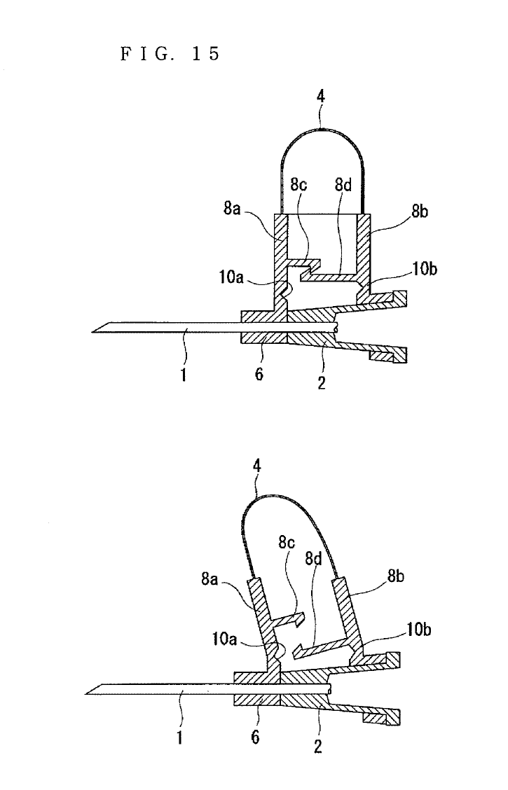

FIGS. 14 to 17 show the sixth embodiment provided with a triggering mechanism.

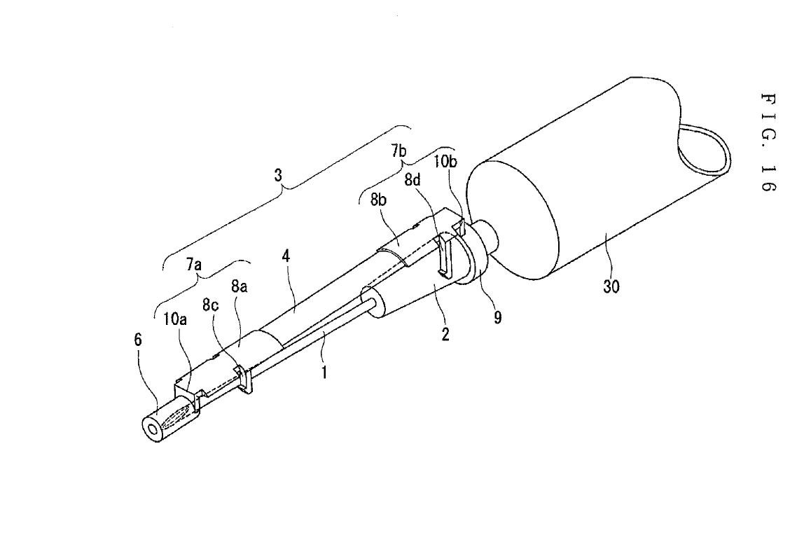

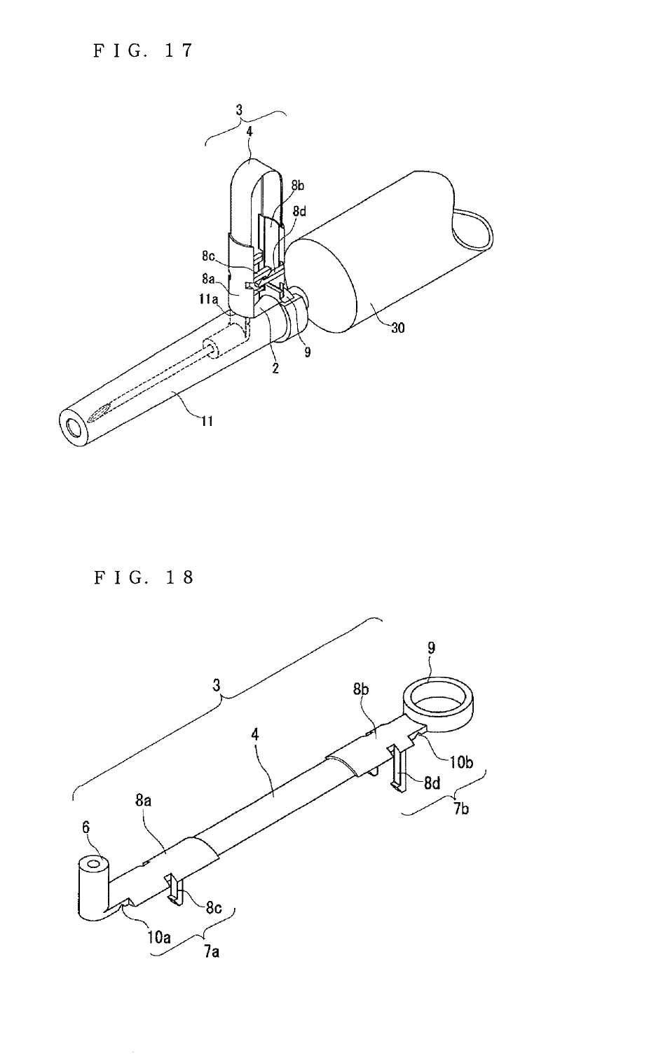

FIG. 14 shows the state immediately before being stuck through the patient's skin. The plate member (4) formed of a metal leaf spring concaves toward needle (1) in cross section perpendicular to the longitudinal direction as clearly shown in FIG. 16.

The plate member (4) is provided a proximal-side-connecting portion (7b) formed of resin for connecting to the needle hub (2) at the proximal side of the plate member (4), and a needlepoint-side-connecting portion (7a) formed of resin connected to the sleeve (6) through which the needle (1) is to be inserted at another end of the plate member (4). The proximal-side-connecting portion (7b) comprises a proximal member (8b) extending from the plate member (4), a fixing ring (9) for fixing on the needle hub (2), and a proximal hinge (10b) for rotatably connecting the proximal member (8b) and the fixing ring (9). The needlepoint-side-connecting portion (7a) comprises a needlepoint member (8a) extending from the plate member (4), and a needlepoint hinge (10a) rotatably connecting the needlepoint member (8a) and the sleeve (6). The needlepoint member (8a) is provided with a needlepoint hook (8c) extending integrally and perpendicularly from the needlepoint member (8a) The needlepoint hook (8c) engages frictionally with a proximal hook (8d) that is also extending perpendicularly from the proximal member (8b). In this state, the sleeve (6) is in the vicinity of the needle hub (2) with a needle (1) inserted.

After use, the medical worker withdraws the injection needle (20) from the patient, and actuates the triggering mechanism. In other words, he/she pushes the bent portion (3) of the plate member (4) toward the needlepoint (1a). Then, as shown in FIG. 15, the needlepoint hook (8c) and the proximal hook (8d) rotate around the needlepoint hinge (10a) and the proximal hinge (10b) respectively in the direction of releasing the frictional engagement. The plate member (4) released from the engaged state changes from the bent state to the extended state by its spring property and extends to the position where the sleeve (6) reaches the position for storing the needlepoint.

Since the triggering mechanism of this embodiment employs a leaf spring as a plate member (4), the needle can be stored by a light operation, and the plate member (4) prevents from being deformed easily by external forces because its geometrical moment of inertia is large. Since it is folded by the needlepoint hook (8c) and the proximal hook (8d) before and during use, the it is made compact as a whole, and it does not disturb the medical procedure by rattling. Since a metal leaf spring is employed as a main structure, it may be constructed of a small number of components as a whole in a simple structure, and thus it can be supplied at low cost. With the reversal-movement-preventing hook (4d) as shown in FIG. 13, the used needle can be prevented from being exposed easily without curving the cross section perpendicular to the longitudinal direction.

FIG. 17 is a drawing showing a state in which a needle-protection cap (11) is mounted. The needle-protection cap (11) is notched at a portion on the side of the opening and provided with a needlepoint-member-receiving portion (11a) that abuts against the needlepoint member (8a). The needlepoint-member-receiving portion (11a) is formed at a position where the frictional engagement between the needlepoint hook (8c) and the proximal hook (8d) does not occur. Therefore, the bending mechanism (3) is prevented from being released accidentally.

In this embodiment, since the needlepoint-member-receiving portion (11a) directly receives the urging force of the plate member (4) in the bent state, the engaging force between the needle-protection cap (11) and the needle hub (2), or between the needle-protection cap (11) and the fixing ring (9) is significantly stronger than the urging force generated by the plate member (4).

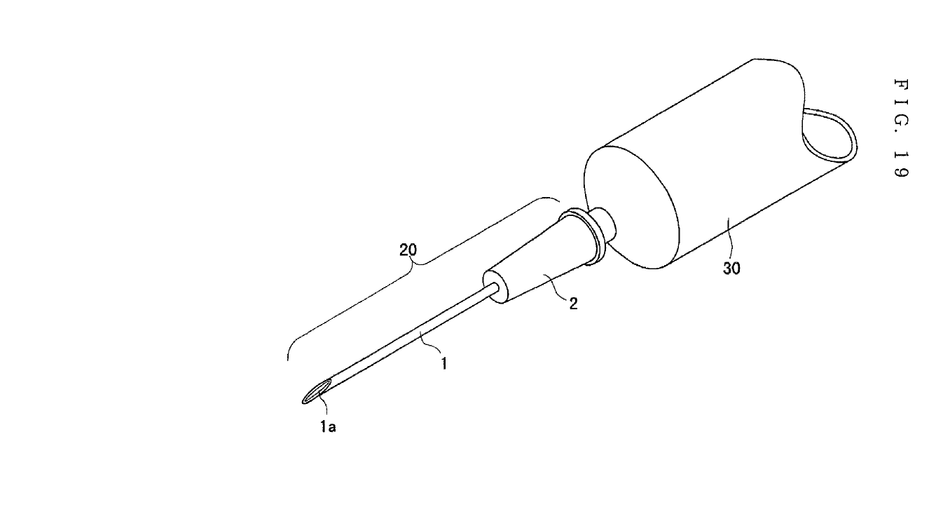

FIG. 18 shows an example of the bending mechanism (3) that can be employed in this embodiment, and shows a simple combination of a pressed part and an injection-molded part. Such bending mechanism (3) is mounted on the conventionally existing and generally used injection needle (20) and the injection cylinder (30) as shown in FIG. 19.

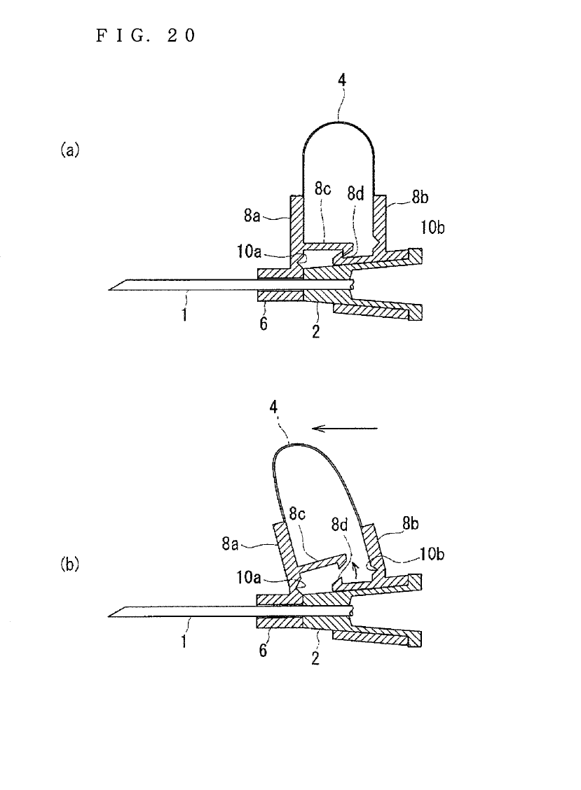

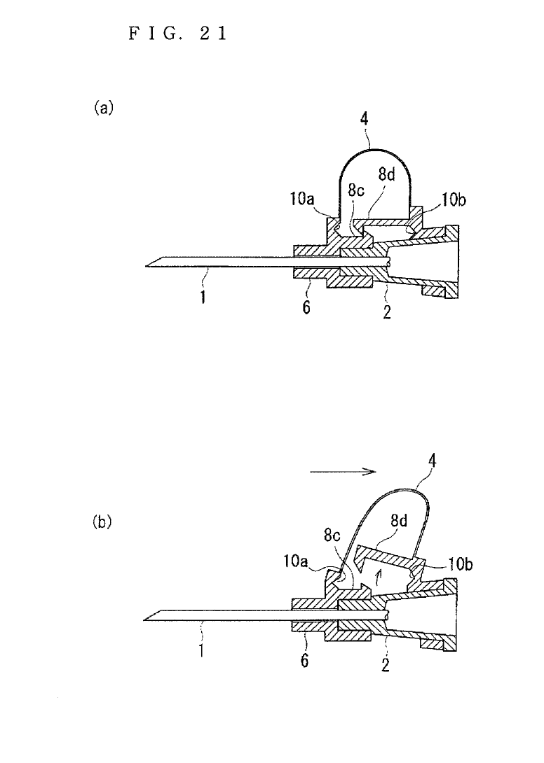

FIGS. 20 and 21 show a modification of the triggering mechanism shown in FIG. 15.

The difference between the triggering mechanism in FIG. 20 and that in FIG. 15 is that the proximal hook (8d) in the triggering mechanism shown in FIG. 20 is formed so as to extend along the needle hub (2).

The difference between the triggering mechanism in FIG. 21 that FIG. 15 is that the needlepoint hook (8c) is formed so as to extend along the sleeve (6), and that the needlepoint hook (8c) is formed upwardly and the proximal hook (8d) is formed downwardly.

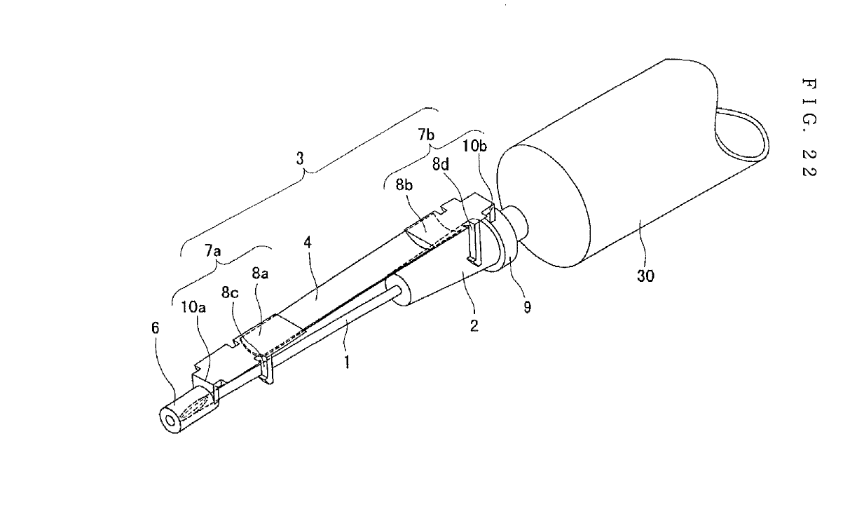

FIGS. 22 to 24 show the seventh embodiment.

In this embodiment, a metal plate member widthwise curved convexly toward the needle (1) is employed as a plate member (4). There is provided a needlepoint hinge (10a) on the side far from the needle (1) with respect to the plate member (4) for rotatably connecting the sleeve (6) and the plate member (4). There is also provided a proximal hinge (10b) on the side far from the needle (1) with respect to the plate member (4) for rotatably connecting the needle hub (2) and the plate member (4).

With the existence of the needlepoint hinge (10a) and the proximal hinge (10b), when external forces in the direction of generation of bending, or external forces that tend to move the sleeve (6) toward the needle hub (2) are exerted on the plate member (4) in the unloaded state, the plate member (4) is bent convexly toward the needle (1). In this case, the existence of the needle (1) limits bending of the plate member (4) to a small extent, and thus the needlepoint (1a) is prevented from being exposed from the sleeve (6) easily.

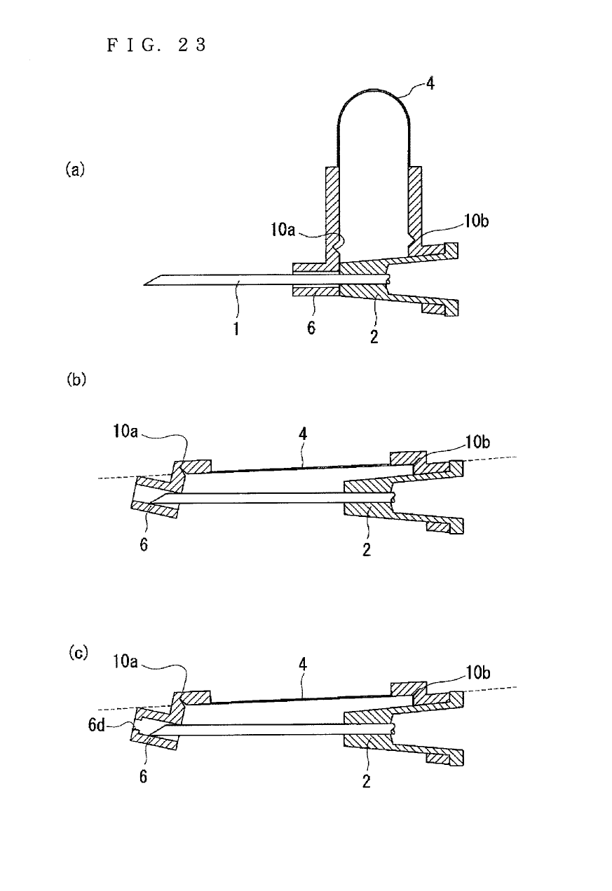

Given that the unloaded state of the needlepoint hinge (10a) is the state before covering as shown in FIG. 23(a), after the needlepoint (1a) is covered by the sleeve (6) as shown in FIG. 23(b), the needlepoint (1a) is constantly kept in contact with the inner wall surface of the sleeve (6) by resiliency of the needlepoint hinge (10a) and is easily getting stuck with the sleeve (6), thereby preventing the needlepoint (1a) from being exposed easily. Providing an inter-wall shoulder (6d) on the inner wall surface of the sleeve (6) to prevent the needlepoint (1a) once stored from being exposed as shown in FIG. 23(c) may increase ease of getting stuck.

In other words, the needlepoint-covering member is provided with a needlepoint-holding portion (4d) positioned in the vicinity of the needlepoint (1a) so that the needlepoint (1a) does not project from the covering portion (6) after the needlepoint (1a) is covered by the covering portion (6).

The “needlepoint-holding portion” may be provided by altering the configuration of the covering portion (6), for example, by forming a shoulder at the portion of the covering portion (6) where the needlepoint (1a) abuts.

Therefore, after the needlepoint (1a) is covered by the covering portion (6), the needlepoint-holding portion (4d) moves closer to the needlepoint (1a) to prevent the needlepoint (1a) from being projected from the covering portion (6). Therefore, the person who handles the injection syringe (20) may be protected from a hazardous needlepoint (1a).

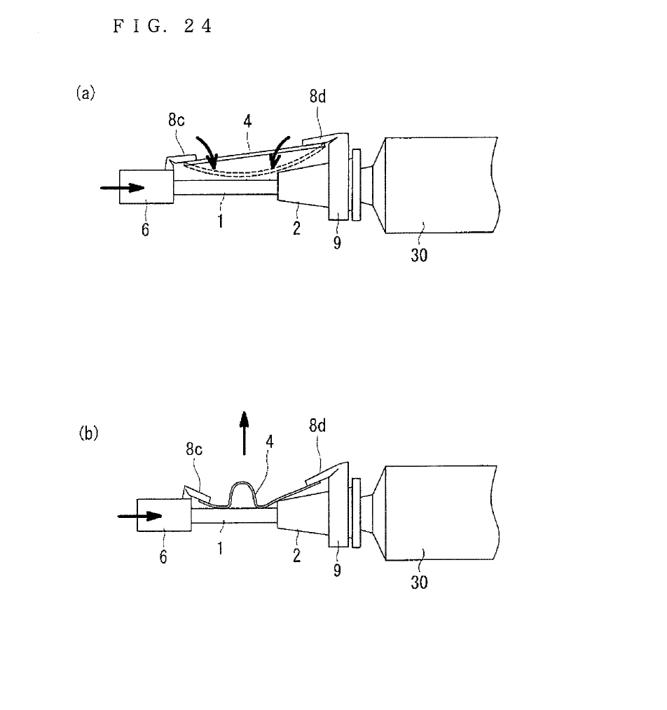

When bending as shown in FIG. 24(b) occurred and further external forces are applied, the portion of the plate member (4) near the center may bend in the direction away from the needle (1), which may result in exposition of the needlepoint (1a). However, in the seventh embodiment, the countermeasure to take a stand against such external forces is not contemplated.

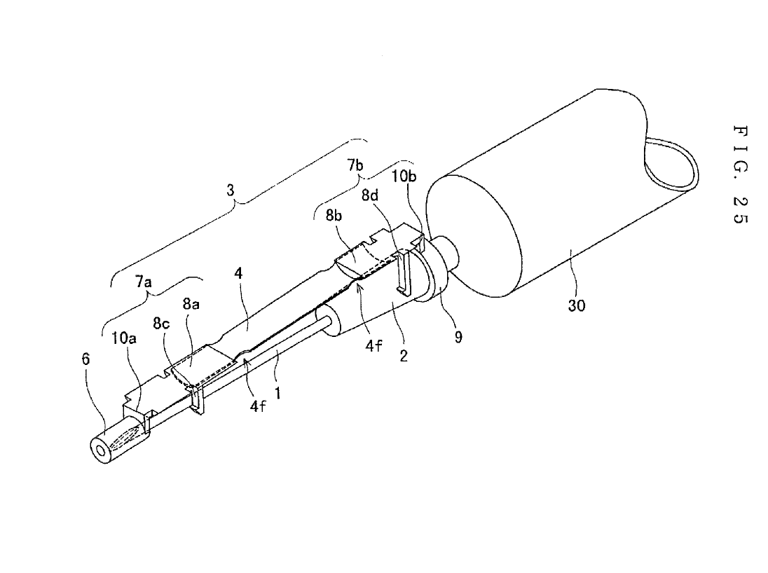

FIG. 25 shows a modification of the seventh embodiment.

This modification differs from the seventh embodiment in that a narrow portion (4f) having a narrower width is formed at the end portion of the plate member (4) adjacent to the connecting portion (7). In other words, the narrow portion (4f) has smaller geometrical moment of inertia in comparison with other portions.

In this modification, when external forces in the direction of generation of bending are exerted on the plate member (4) in the extended state, torque is produced at the connecting portion (7) in the direction to curve the plate member (4) convexly toward the needle (1). Since geometrical moment of inertia is smaller at the narrow portion (4f) adjacent to the connecting portion (7) in comparison with other portions, bending occurs at the narrow portion (4f), and the connecting portion (7) rotates around the hinge to be brought into contact with the needle (1) along with the end portion of the plate member (4).

When external forces further increase, a compressive load applied longitudinally to the plate member also increases and concurrently bending moment from the connecting portion (7) to the needle (1) increases as well. Therefore, bending load applied toward the plate member (4) is distributed, and thus other portion are not bent easily, whereby the needlepoint (1a) is maintained in the state being covered by the sleeve (6).

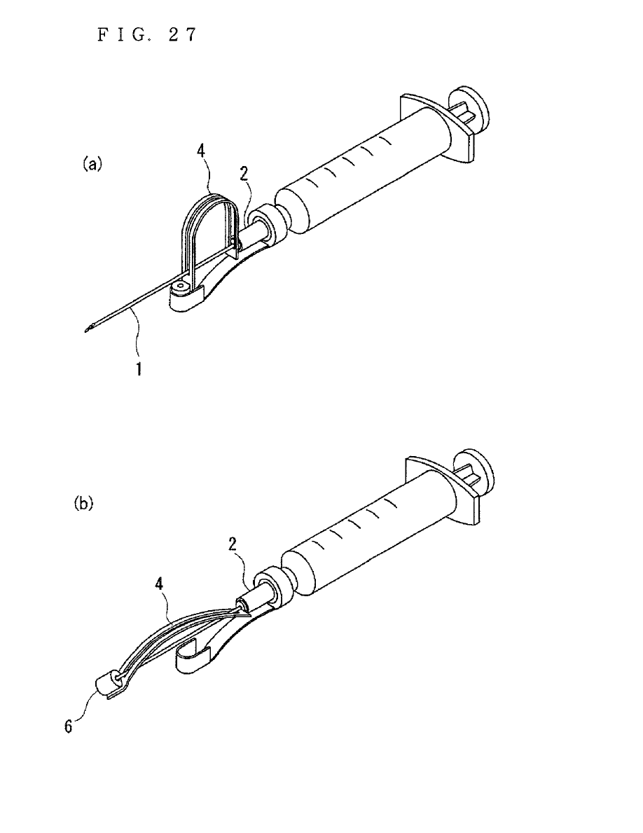

FIGS. 27 to 29 show the eighth embodiment.

FIG. 27 shows that the plate member (4) is provided with an insertion groove extending between both ends of the plate member (4) for allowing the needle (1) to pass through the direction of the thickness. The plate member (4) is fixed in such a manner that the root of the needle (1) is positioned at an end of the insertion groove of the plate member (4). The plate member (4) has such a length that the sleeve (6) reaches the position of the needlepoint (1a), and allows the needlepoint (1a) to project from another end of the insertion groove when released from the load. When the trigger member (not shown) released the state of deformed into a U-shape, the sleeve (6) moves toward the needlepoint (1a) and protect the needlepoint (1a).

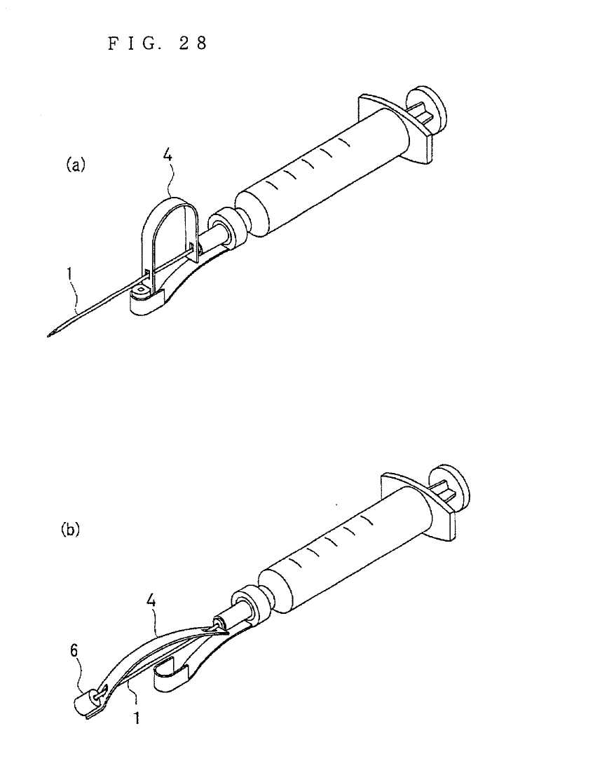

FIG. 28 is a modification of the embodiment shown in FIG. 27, in which two insertion holes are provided at the positions in the vicinity of both ends instead of the insertion groove.

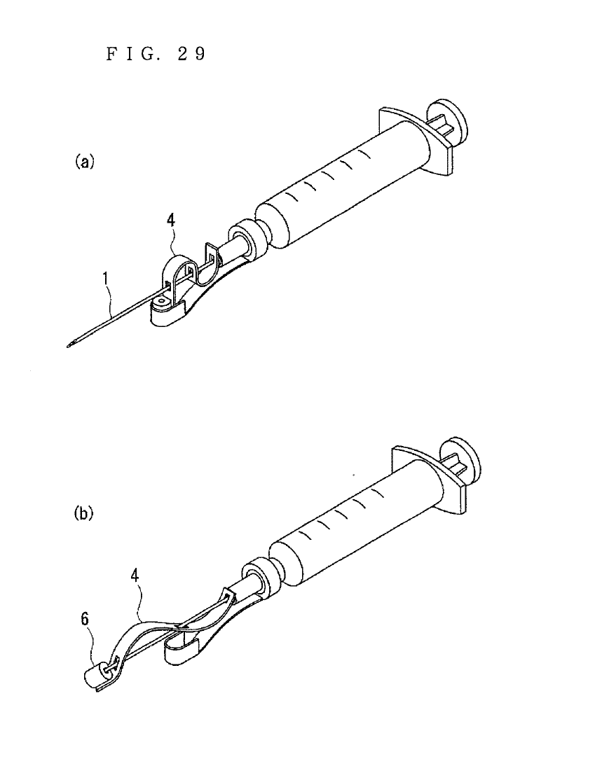

FIG. 29 shows a modification of the embodiment shown in FIGS. 27 and 28. The plate member (4) is deformed in an S-shape, and is provided with insertion holes at both ends and at the center near the inflexion point. This modification can realize the most compact structure.

As described thus far, this embodiment is a needlepoint-covering member for covering the needlepoint (1a) of the injection needle (20) provided with a needle (1) having a sharp needlepoint (1a) and a needle hub (2) to be connected to the cylinder located at an end of the needle (1) far from the needlepoint (1a) comprising a plate member (4) fixed at one end to the needle hub (2) side and being bendable to maintain the state before covering, and a covering portion (e.g. the sleeve 6) in a tubular shape having a bottom for covering the needlepoint (1a) when the plate member (4) in the bent state is extended, wherein the plate member (4) has such a length that the covering portion (6) reaches the position of the needlepoint (1a), and is provided with insertion holes at least in the vicinity of both ends of the plate member (4) for allowing the needle (1) to pass through in the direction of the thickness, wherein the covering portion (6) is disposed at the position nearer to the needlepoint (1a) than the insertion hole on the needlepoint (1a) side.

The “insertion hole” must simply be disposed at least in the vicinity of both ends of the plate member (4). For example, it may be two insertion holes provided in the vicinity of both ends, an insertion groove extending between both ends, and three insertion holes provided at both ends and at the inflection point so that the plate member (4) is longitudinally curved in an S-shape before covering.

Therefore, the plate member (4) is fixed at one end to the needle hub (2) side. Since the plate member (4) is formed to be deformable by bending, it can exhibit the bent state and the extended state. In other words, when the plate member (4) is in the extended state, the covering portion (6) covers the needlepoint (1a). Since the covering portion (6) has a bottom in a tubular shape, the needlepoint (1a) cannot be exposed unless the bottom is broken.

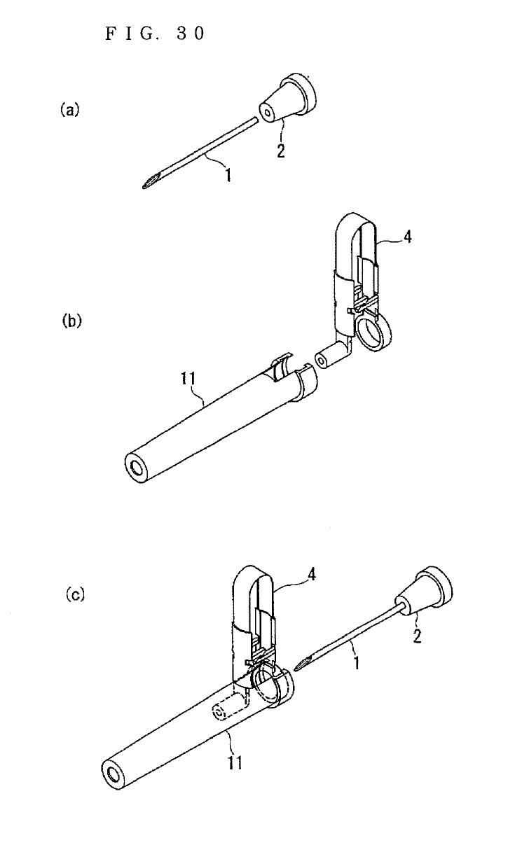

FIG. 30 shows a method of assembling the injection needle with a needlepoint-covering member.

As a first step, the needle (1) and the needle hub (2) are fixed for manufacturing a hub with a needle. In the second step, the needlepoint-covering member is centered and built in the cap (11) for covering the needle (1). Then the hub with a needle is built in the cap (11) that is provided with the needlepoint-covering member incorporated in the incorporating step.

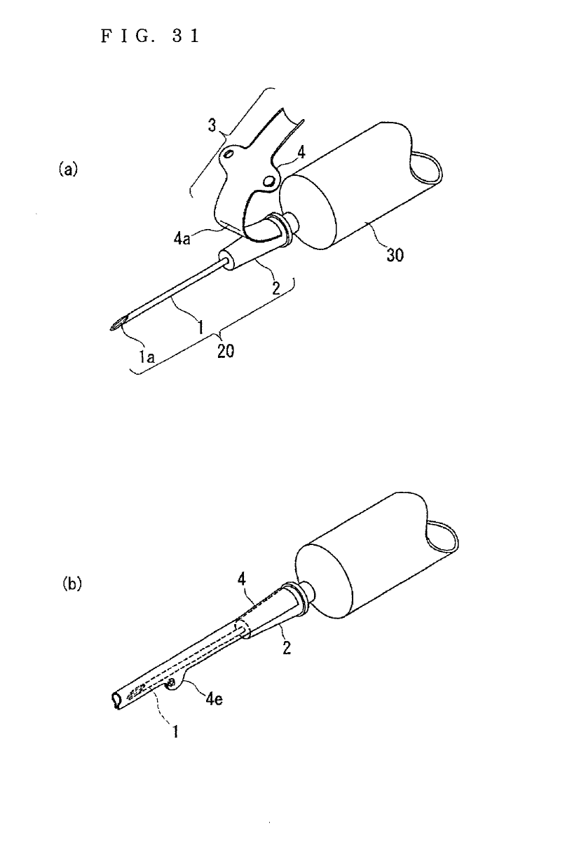

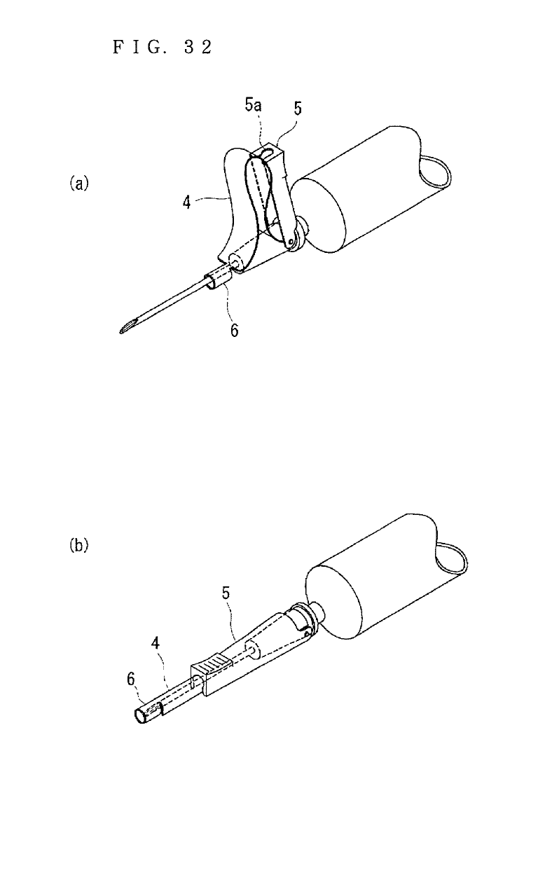

FIGS. 31 to 33 show the ninth embodiment.

FIG. 31 shows a plate member (4) in the embodiment shown in FIG. 1 provided with a “wrap-around fixing portion (4e)” in the vicinity of its central position. The wrap-around fixing portion (4e) is formed to decrease the curvature radius of the widthwise curve of the plate member (4) concaved toward the needle (1) when it wraps around the circumference of the needle (1).

After the needlepoint (1a) is covered by the plate member (4), the wrap-around fixing portion (4e) wraps around and fixes the circumference of the needle (1) near the longitudinal center of the needle (1). Then, the wrap-around fixing portion (4e) decreases the curvature radius of the plate member (4) that is widthwise concaved toward the needle (1), so that geometrical moment of inertia against external forces that tend to remove the plate member (4) from the needlepoint (1a) increases, and thus the plate member (4) is unified with the needle (1), thereby preventing the needlepoint (1a) from being exposed.

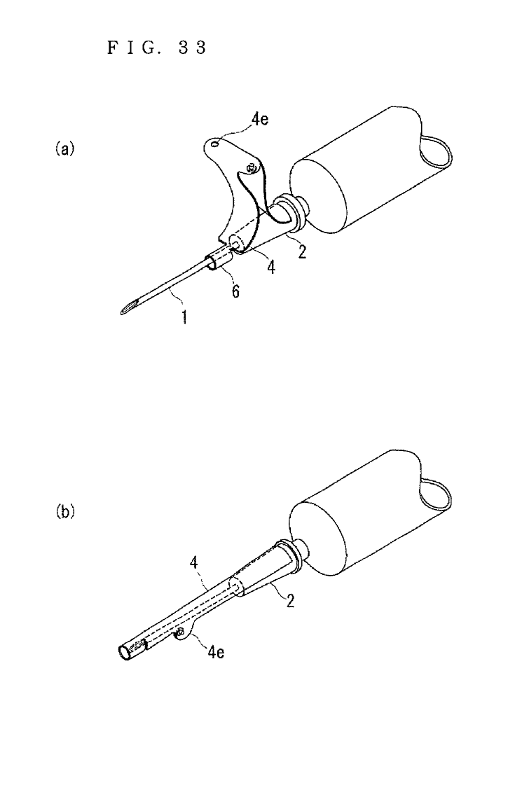

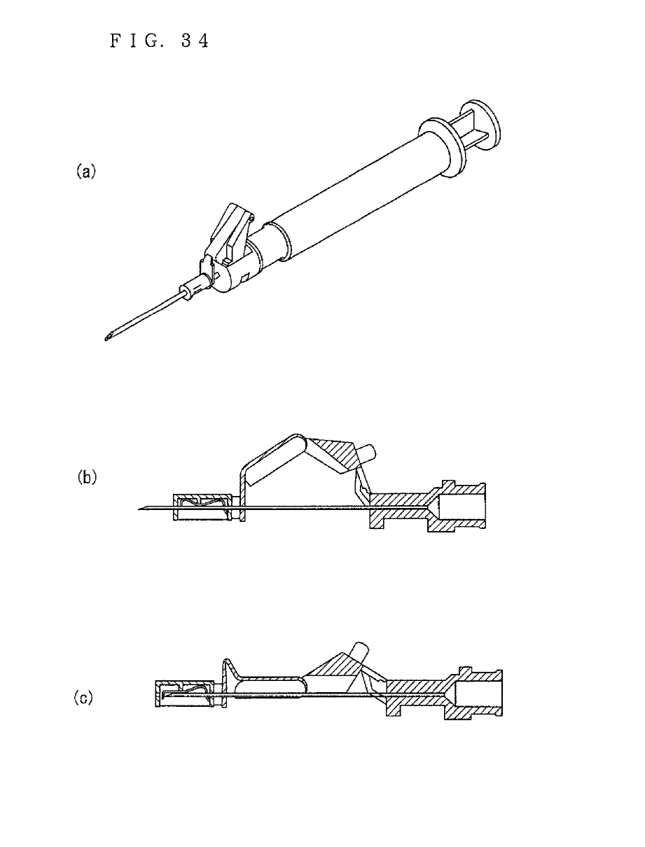

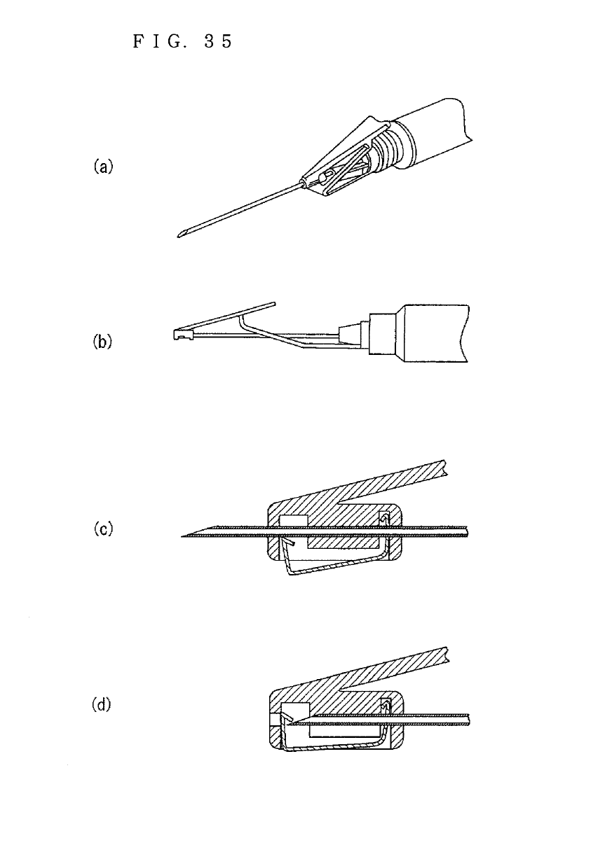

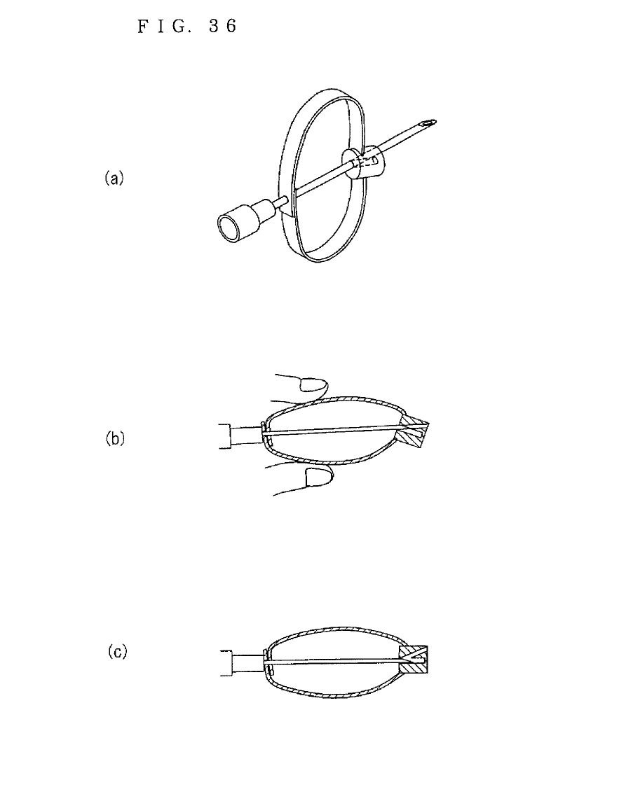

FIG. 32 shows a modification of the embodiment shown in FIG. 8. This modification is provided with an operating member (5) in a box-shaped mounted at one end on the needle hub (2) so as to swing with respect to the needle hub (2) and is opening toward the needle hub (2) side. The surface facing toward the needlepoint (1a) of the operating member (5) is formed with a mold hole (5a) for forcing the plate member (4) to be widthwise concaved toward the needle (1). The plate member (4) is also provided at its tip with a tubular covering portion (6), a part of which is linked to the plate member (4).

After use of the needle (1), the operating member (5) is moved down toward the needle hub (2). Then, the covering portion (6) reaches and covers the needlepoint (1a). The plate member (4) is deformed along the shape of the mold hole (5a) formed on the operating member (5). Accordingly, geometrical moment of inertia against external forces that tend to remove the covering portion (6) from the needlepoint (1a) increases. Since there is a non-continuous portion between the covering portion (6) and the plate member (4), larger external forces being exerted, the continuous portion between the covering portion (6) and the plate member (4) deforms, thereby preventing the needlepoint (1a) from being exposed.

FIG. 33 shows a modification of the embodiment shown in FIG. 31. This modification is not provided with the operating member (5), but provided with a “wrap-around fixing portion (4e)”, and the plate member (4) is provided at its tip with a cylindrical covering portion (6) a part of which is linked to the plate member (4). In this modification as well, after the needlepoint (1a) is covered by the covering portion (6), the wrap-around fixing portion (4e) wraps around the needle (1) near the longitudinal center of the needle (1) and fixes the needle (1). Then, since the wrap-around fixing portion (4e) decreases the curvature radius of the plate member (4) that is widthwise concaved toward the needle (1), geometrical moment of inertia against external forces that tend to remove the covering portion (6) from the needlepoint (1a) increases, and thus the plate member (4) is unified with the needle (1) to prevent the needlepoint (1a) from being exposed. Since there is provided a non-continuous portion between the covering portion (6) and the plate member (4), when larger external forces are exerted, the continuous portion between the covering portion (6) and the plate member (4) deforms and prevents the needlepoint (1a) from being exposed.

The present invention provides a novel needlepoint-covering member in which the flow of medical procedure is not disturbed before used as an injection needle, the tip of the injection needle is suitably protected after use of the injection needle, and the protecting member cannot be removed easily even when external forces are exerted.

The present invention further provides a method of assembling an injection needle with a needlepoint-covering member.

The present invention further provides a novel injection needle with a needlepoint-covering member in which the flow of medical procedure is not disturbed before used as an injection needle, the tip of the injection needle is suitably protected after use of the injection needle, and the protecting member cannot be removed easily even when external forces are exerted.

What is claimed is:

1. A needlepoint-covering member for covering the needlepoint of the injection needle comprising a needle having a sharp needlepoint and a needle hub provided at the end of the needle far from the needlepoint for being joined with the cylinder, comprising:
a plate member whose end is fixed to the needle hub side and which can be bent to less than half of the axial length in the extended state in order to maintain the state before covering; and
a covering portion for covering the needlepoint when the bent plate member is extended;
wherein the plate member has a length to allow the covering portion to reach the position of the needlepoint when extended; and is widthwise concaved toward the needle for resisting external forces that tend to remove the covering portion that covers the needlepoint from the needlepoint.

2. A needlepoint-covering member according to claim 1, wherein the plate member is provided with a wrap-around fixing portion for embracing the circumference of the needle and fixing the needle in the vicinity of the center in the axial direction of the plate member, and the wrap-around fixing portion is formed in such a manner that the curvature radius of the plate member that is widthwise concaved toward the needle is decreased when the circumference of the needle is embraced and fixed thereby.

3. A needlepoint-covering member according to claim 1, wherein the covering portion is a tubular body comprising an insertion hole through which the needle and the plate member are inserted, and the engaging portion for engaging with the plate member in a state where the covering portion reaches the needlepoint; and
wherein the plate member is provided with an engaged portion for engaging with the engaging portion.

4. A needlepoint-covering member for covering the needlepoint of the injection needle comprising a needle having a sharp needlepoint and a needle hub provided at the end of the needle far from the needlepoint for being joined with the cylinder, comprising:
a plate member whose end is fixed to the needle hub side and which can be bent to less than half of the axial length in the extended state in order to maintain the state before covering; and
a covering portion for covering the needlepoint when the bent plate member is extended;
wherein the covering portion has a tubular shape for allowing the needle to pass through when the plate member is bent;
wherein the plate member has a length to allow the covering portion to reach the position of the needlepoint when extended; and
wherein the direction in which the plate member is bent by external forces that tend to remove the covering portion from the needlepoint is convex toward the needle.

5. A needlepoint-covering member according to claim 4, wherein the plate member is formed of a plate member that is widthwise curved convexly toward the needle.

6. A needlepoint-covering member according to claim 4, wherein geometrical moment of inertia of at least one of both ends of the plate member in the extended state is determined to the value lower than that of other portions.

7. A needlepoint-covering member according to claim 4, further comprising a needlepoint hinge for rotatably connecting the covering portion and the plate member on the side far from the needle with respect to the plate member.

8. A needlepoint-covering member according to claim 4, further comprising a proximal hinge for rotatably connecting the needle hub and the plate member on the side far from the needle with respect to the plate member.

9. A needlepoint-covering member according to claim 1, further comprising:
a bending-position-maintaining member for maintaining the plate member in the bent state before covering the needlepoint by the covering portion, and
a trigger-function portion for releasing a load from the state of being maintained in the bent state.

10. A needlepoint-covering member according to claim 4, further comprising:
a bending-position-maintaining member for maintaining the plate member in the bent state before covering the needlepoint by the covering portion, and
a trigger-function portion for releasing a load from the state of being maintained in the bent state.

11. A needlepoint-covering member according to claim 1, wherein the plate member comprises a proximal hinge for fixing the end on the needle hub side of the plate member so as to be capable of rotating with respect to the axis of the needle; an upwardly opened proximal hook fixed on the plate member at the position closer to the needle hub; and a downwardly opened needlepoint hook fixed on the plate member at the position closer to the needlepoint;
wherein the plate member is maintained in the bent state by engaging the proximal hook and the needlepoint hook, and the engaged state between the proximal hook and the needlepoint hook may be released by changing the angle of the proximal hinge.

12. A needlepoint-covering member according to claim 4, wherein the plate member comprises a proximal hinge for fixing the end on the needle hub side of the plate member so as to be capable of rotating with respect to the axis of the needle; an upwardly opened proximal hook fixed on the plate member at the position closer to the needle hub; and a downwardly opened needlepoint hook fixed on the plate member at the position closer to the needlepoint;
wherein the plate member is maintained in the bent state by engaging the proximal hook and the needlepoint hook, and the engaged state between the proximal hook and the needlepoint hook may be released by changing the angle of the proximal hinge.

13. A needlepoint-covering member according to claim 11, wherein the plate member comprises a needlepoint hinge for fixing the end portion on the needlepoint side of the plate member so as to be capable of rotating with respect to the axis of the needle.

14. A needlepoint-covering member according to claim 12, wherein the plate member comprises a needlepoint hinge for fixing the end portion on the needlepoint side of the plate member so as to be capable of rotating with respect to the axis of the needle.

15. A needlepoint-covering member according to claim 1, further comprising a cap for covering at least the needlepoint and the needle with the plate member maintained in the bent state.

16. A needlepoint-covering member according to claim 4, further comprising a cap for covering at least the needlepoint and the needle with the plate member maintained in the bent state.

17. A method of assembling the injection needle with a needlepoint-covering member comprising:
a fixing step for fixing the needle and the needle hub for manufacturing a hub with a needle;
an incorporating step for centering and incorporating the needlepoint-covering member into a cap for covering the needle; and
a capping step for incorporating the hub with a needle into the cap that is provided with the needlepoint-covering member incorporated in the incorporating step.

18. An injection syringe with a needlepoint-covering member described in claim 1, comprising:
a hub with a needle in which a needle and a needle hub are fixed with each other, and
an injection syringe provided with the hub with a needle fixed at its tip.

19. An injection syringe with a needlepoint-covering member described in claim 4, comprising:
a hub with a needle in which a needle and a needle hub are fixed with each other, and
an injection syringe provided with the hub with a needle fixed at its tip.
